(19) United States
(12) Patent Application Publication
(10) Pub. No.: US 2007/0239169 A1
(43) Pub. Date: Oct. 11, 2007

(54) REFERENCE MARKER AND USE IN A MOTION TRACKING SYSTEM

(75) Inventors: Christopher Plaskos, New York, NY (US); Stephane Lavallee, Saint Martin d'Uriage (FR)

Correspondence Address: DARBY & DARBY P.C., P.O. BOX 770, Church Street Station, New York, NY, 10008-0770, US

(73) Assignee: Perception Raisonnement Action en Medecine, La Tronche (FR)

(21) Appl. No.: 11/687,324

(22) Filed: Mar. 16, 2007

Related U.S. Application Data
(60) Provisional application No. 60/783,565, filed on Mar. 17, 2006.
(60) Provisional application No. 60/889,366, filed on Feb. 12, 2007.

(51) Int. Cl. A61B 17/60; A61B 5/05

(52) U.S. Cl. 606/96; 600/424

(57) ABSTRACT

The present invention is directed to reference markers that are used in position measuring device that can be part of a computer assisted surgery (CAOS) system. The reference marker is constructed of a rigid body that has 360 degrees of visibility.

### CROSS-REFERENCE TO RELATED APPLICATIONS

The present application claims the benefit of U.S. patent application Ser. Nos. 60/783,565, filed Mar. 17, 2006, and 60/889,366, filed Feb. 12, 2007, both of which are hereby expressly incorporated herein by reference in their entirety.

### TECHNICAL FIELD

The present invention relates generally to motion tracking with a position measuring device, such as optical cameras, and is particularly applicable to computer assisted surgery systems, as well as, other applications, such as, those in medical or industrial related fields in which motion tracking is performed.

### BACKGROUND

The principle of localization by stereovision is coupled to calibration, pairing, or triangulation. The simplest object that is localized by a stereovision system is a sphere or a disc. Conventionally known as markers, they are manufactured in plastic and covered with a reflective paper. This type of a marker suffers from several drawbacks. First, covering a sphere with paper is not easy accomplished which drives the cost up. Secondly, spheres are not reliable. The marker can be partially occluded by other objects and information missing in the image will imply error in localization. Thirdly, at least 3 markers are needed to completely localize (six degrees of freedom (6 DOF)) an object. The size is not optimized.

Many designs exist for localization by pattern; however, they require a combination of flat surfaces. This approach also suffers from several drawbacks. Namely, being that the pattern is a flat surface and therefore, the angle of visibility is limited. To increase the angle of visibility, several patterns must be combined together in different planes. This results in increased complexity in design and in manufacturing.

### SUMMARY

The present invention is directed to reference markers that are used in a position measuring device that can be part of a computer assisted surgery (CAOS) system. The reference marker is constructed of a rigid body that has up to 360 degrees of visibility.

According to one exemplary embodiment, a rigid body with a substantially increased range of visibility includes four reflective spheres arranged in a pyramid formation such that four distinct planes (faces) are defined. The rigid body also includes disc shaped dividers arranged to prevent spheres from behind from overlapping the spheres in the front when the spheres from behind are in line with the line of sight of a detector.

According to one exemplary embodiment, a rigid body with a substantially increased range of visibility includes four reflective spheres arranged in a pyramid formation such that up to four distinct reference planes (faces) are defined. Each reference plane is defined by three spheres. The rigid body includes disc-shaped dividers arranged to prevent spheres from behind from overlapping the spheres in the front when the spheres from behind are in line with the line of sight of a detector.

In another aspect of the present invention, a motion tracking system includes a position measuring device for detecting the position and orientation of an object by tracking the relative movement of a reference marker attached to the object. The system includes a computer that is configured to determine and track positions of the reference body. The reference marker is constructed so that it includes four distinct reference faces that are arranged such that the reference marker has 360 degrees of visibility since at least one reference face is always in view and is visible to the position measuring device.

### BRIEF DESCRIPTION OF THE DRAWINGS

The foregoing and other features of the present invention will be more readily apparent from the following detailed description and drawing figures of illustrative embodiments of the invention in which:

FIG. 1 is a schematic of a computer-assisted orthopedic surgery (CAOS) system according to one embodiment;

FIG. 2 is a perspective view of a reference marker (RM) shown attached to a distal femur bone in accordance with an embodiment of the present invention;

FIG. 3 is a perspective view of a low profile base that is used with the reference marker for installing it to the distal femur bone;

FIGS. 4A and 4B are perspective views of a reference marker shown attached to the distal femur bone in accordance with one embodiment of the present invention;

FIGS. 5A and FIG. 5B are perspective views of a reference marker shown attached to the distal femur bone according with one embodiment of the present invention;

FIG. 5C is two-dimensional image of the reference marker of FIGS. 5A and 5B;

FIGS. 6-8 are perspective views of a rigid body with 360 degrees of visibility according to one exemplary embodiment of the present invention;

FIGS. 9-10 are perspective views of a rigid body with 360 degrees of visibility according to another exemplary embodiment; and

FIG. 11 is a perspective view of a rigid body with 360 degrees of visibility according to yet another exemplary embodiment.

### DETAILED DESCRIPTION OF CERTAIN EMBODIMENTS

By way of overview, the present invention provides an improved reference marker for motion tracking with optical cameras and is suitable for computer assisted surgery (CAOS) systems, as well as other applications, such as, those in medical or industrial related fields in which motion tracking is performed.

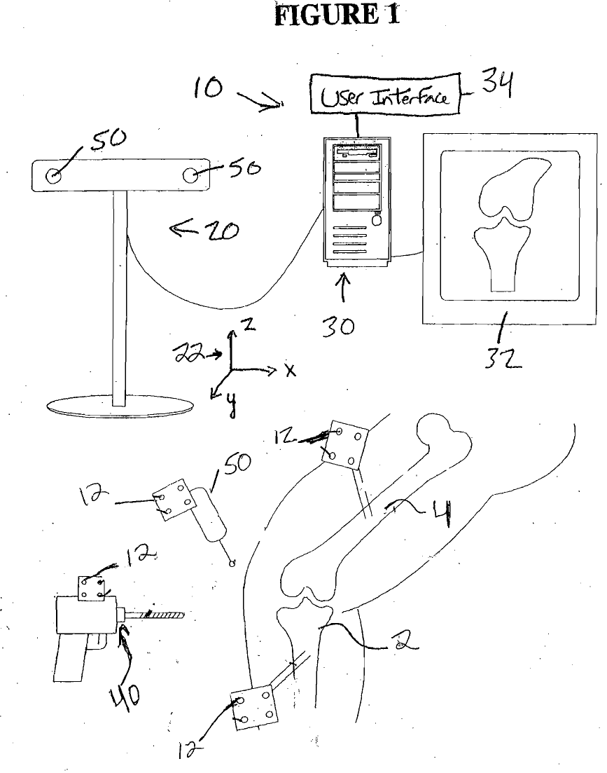

More specifically and as shown in FIG. 1, a CAOS system 10 typically includes a position measuring device 20 that can accurately measure the position of marking elements in a three dimensional space (represented by coordinate system 22). The position measuring device 20 can employ any type of position measuring method as may be known in the art, for example, emitter/detector or reflector systems including optic, acoustic or other wave forms, shape based recognition tracking algorithms, or video-based, mechanical, electromagnetic and radio frequency systems. In one embodiment, the position measuring device 20 is preferably an optical tracking system that includes at least one camera 50 that is in communication with a computer system 30 and positioned to detect light reflected from a number of special light reflecting markers or spheres 12. When a pair of cameras 50 are used, these cameras can be 2D cameras that use a model, such as the Direct Linear Transform (DLT) Method to obtain 3D information on the position of a single marker such as a sphere in space. Alternatively, three 1D cameras can arranged relative to each-other and used to obtain 3D information on the position of a single marker such as a sphere in space. Many different configurations of multiple cameras can be used.

Detecting and determining the position and orientation of an object is referred to herein as “tracking” the object. To provide precision tracking of objects, reference markers 12 can be rigidly connected together to form reference bodies which can be attached to bones (such as, tibia 2 and femur 4), tools and other objects to be tracked as described in more detail below. Examples of such devices that have been found to be suitable for performing the tracking function include the Polaris™ and Optetrak™ systems from Northern Digital Inc., Ontario, Canada.

Exemplary position measurement devices 20 and associated methods of use are described in greater detail in a number of publications, including U.S. Pat. Nos. 5,564,437; 5,828,770; 6,351,659; and 5,834,759; and United States patent application publication No 2005/0101966 by S. Lavallee, all of which are incorporated by reference in their entirety.

The relative position of the patient's bones, such as the patient's tibia 2 and the patient's femur 4, can be determined and tracked by attaching reference bodies which include respective markers 12. The reference bodies can also be labelled or shaped in the form of numbers (e.g., “1”, “2”, “3” . . . ) or alphabetical letters, such as, “F” for Femur, “T”for tibia, “P” for pointer, and so on, so as to avoid confusion as to which reference body should be attached to which bone or tool.

The tracked objects and there relative positions can be displayed on a screen 32 that is connected to the computer system 30. In an embodiment, the display is a touch screen which can also be used for data entry and/or a user interface 34 is provided.

The position measurement device similarly can include a number of different tools that are used at different locations and perform different functions as the system is operated to yield optimal data and information. These tools include the above described markers 12, which act as landmark markers, as well as, other tools, such as a milling or burring device or cutting tool 40 having a number of markers 12, which is an example of an object trackable by position measuring device. The system also includes a pointer 50, with respective markers 12, which can be used to digitize points on the surfaces of the target object, which can be bone, such as, the femur 4, tibia 2 or pelvis or it can be another object.

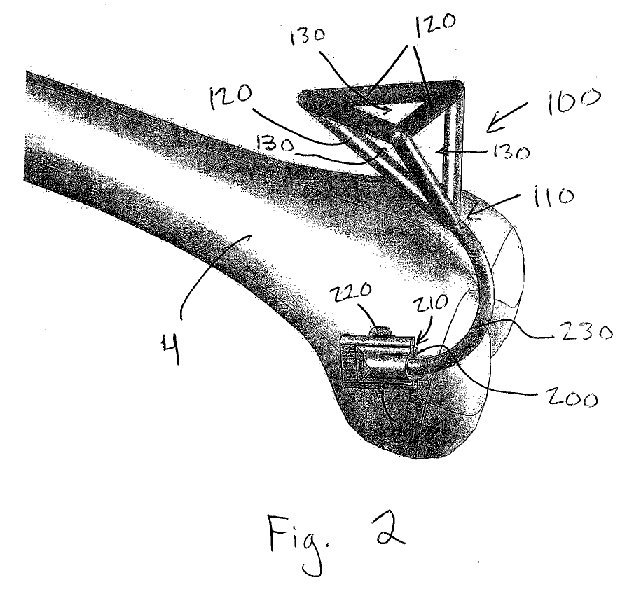

Typically, the reference marker 12 can be formed of a cylinder (cylindrical body) which serves as a basic shape for the marker. The cylinder is covered by a reflective material, coating, film, sticker, paper, etc . . . and produces a line in an image. A line in one 2D image gives one 3D plane, e.g., 2×cameras→2×3D planes→1×3D line. At least 2 lines are needed to completely localize (6 DOF) an object. The line need not be infinite and a line segment is sufficient for this purpose. The length of the segment can be used for identification only. The above arrangement is easily manufacturable by just rolling a plastic cylinder in a reflective material or paper. The paper need not be deformed, and can be rolled onto each cylinder or strut individually either before or after the cylinders are assembled together to form the reference marker, depending on how the reference marker is manufactured. Robust detection to partial occlusion is accomplished in a compact design, ratio information versus size of the frame is improved, and with 3 times the amount of lines, better angle of visibility is accomplished. Referring now to FIG. 2, a reference marker (RM) 100 according to one exemplary embodiment of the present invention is shown attached to the distal femur 4. The reference marker 100 is formed of a body 110 that can be made up of a series of bars 120 to make a 3D shape. One possible shape for the reference body can be a pyramid or triangular shape, in which each pyramid strut is made from cylinders or bars 120. A pyramid with a triangular base has 3 triangular sides 130. Each bar 120 is reflective to light and in particular, to infrared light. This can be achieved using a number of methods, for example, applying a reflective film such as those marketed by the company 3M. Alternatively, the entire structure 100 can be dipped in reflective paint or spray painted, or it can be made out of a reflective material. The RM 100 can be made out of injection molded plastic, and can be a single use product, i.e., disposable. In fact, the entire structure as defined by the RM 100, a support 230, and a base 200 can be made from plastic or any other economic material so that it can be disposed of at the end of the surgery, thereby reducing the risks associated with sterilization and costs associated with cleaning.

Known algorithms can be used to detect the position of each strut in the images of the camera, such as edge detection images, ‘Canny’ operators, or Hough Transforms, thresholding techniques, etc. ‘Edge detection’, From Wikipedia, the free encyclopedia, available at http://en.wikipedia.org/wiki/Edge_detection and ‘Edge Detection Techniques An Overview’ by Djemel Ziou, Salvatore Tabbone published in the International Journal of Pattern Recognition and Image Analysis (1998) disclose such techniques and are hereby incorporated by reference in their entirety.

Each reference face (RF) 130, which in this triangular is a triangular face, of the reference marker 100 (RM) can be seen as a reference frame detected by the camera. Such camera systems are well known and described for computer aided surgery. A reference frame can be constructed from each face 130 using the three bars 120 detected. Only two lines are required to construct a reference frame, for example, by taking the cross product of the two vectors A and B, to obtain a new vector C which is perpendicular to vectors A and B. Taking the cross product again with C and A or B, thus makes an orthogonal reference frame. Note that three bars 120 are included in each reference face 130, thus we have additional information. This can help improve accuracy and robustness, by for example, using averaging techniques. In addition, often multiple reference faces 130 and reference frames are in view. Since the geometry of each RM 100 and RF 130 is known, this relationship can be exploited to yet again further improve the accuracy and reduce the measurement noise. This can be accomplished using a number of methods, such as, for example, averaging methods. In addition, weighting can be added to each RF 130 or even each strut of each RF 130 to take into account its visibility. For example, if one strut is occluded by the orientation of the RM 100 with respect to the camera, this RF 130 can have a lower weighting than another more visible RF 130. Struts can also get covered in blood or other such fluids during surgery, and the reference face 130 and reference frames can still be reconstructed since the other struts are still visible.

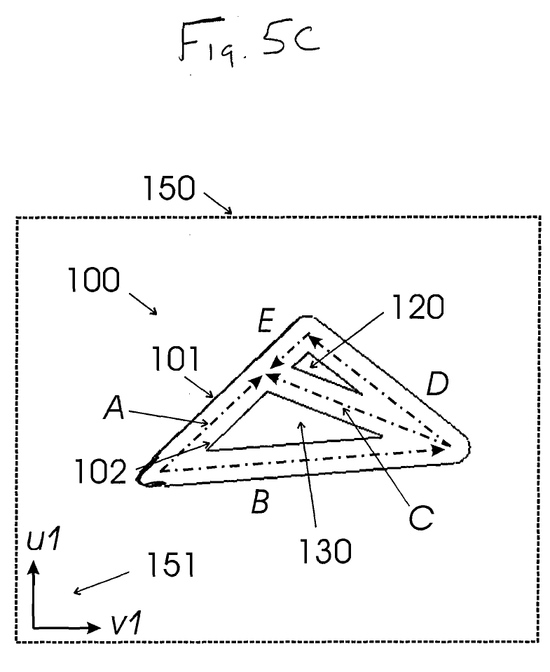

Referring now to FIG. 5C, a two-dimensional (2D) image 150 with pixel co-ordinate system [u1, v1] (151) containing a view of RM 100 as might be seen from one of the cameras 50. Lines A, B and C are extracted from the struts forming RF 130, and lines C, D, and E are extracted from the struts forming RF 120. Line A can be determined in image 150 by detecting adjacent edges 101 and 102, and then determining the centerline between them (i.e., the line of symmetry between edges 101 and 102). Edge detection techniques described above can be used to detect the edges of each strut. Thus, line A is known in co-ordinate system [u1,v1] image. All other lines in image 150 can be calculated. Similarly, using a second image from the second camera taken at the same time but having a different line of sight from first camera, lines A to E can also be constructed in the second image and there positions known in the second pixel co-ordinate system [u2,v2]. The relative positions of both cameras and the transformations between their respective pixel co-ordinates systems can pre-calibrated and known. The planes in 3D space corresponding to the each line in each camera image view can be calculated, and their intersections can be calculated to determine the position of vectors A to E in the 3D space of the camera. Vectors A to E correspond to the centerline or center axis of each individual struts.

RM 100 is preferably designed such that all RF's have a different geometry from one another. In particular, the angle between any vector pair in a RF (i.e., neighboring vectors AB constitute a pair) can be different and unique from any other vector pair in that RF, and from any other pair in any other RF of the RM. Thus, RF 130 could have angles of 55°, 60°, and 65° between vector pairs AB, BC, and CA, respectively. RF 120 could have angles of 35°, 45°, and 90° between vector pairs CD, DE, and EC, respectively. Similarly, unique angles could be assigned to all other vector pairs such as AE, BD, EF and so on (note vector F shown). Preferably the minimum difference between the angels is chosen to be sufficiently large such that the camera resolution is high enough to robustly determine which angle belongs to which vector pair. Since each vector pair has a unique angle associated to it, and the full geometry of the RM is known and pre-stored in the computer memory, only one vector pair need be visible to determine the 6DOF position and orientation information to fully track the RM 100. Assume now that RM 100 is a particular orientation such that not all struts of a particular RF are visible. For example, strut C is in front of and occludes struts D and/or E. In this case it may be difficult to accurately determine Vectors C, D, and E because of the overlapping edges in the image. Vectors A and B however are visible and their 3D positions and the angle between them can be calculated. This angle can be related back the stored geometry file of the RM, and the complete 6 DOF position of the RM can be determined. Even if the RM is partially covered by an object such as a hand or tool or by blood, only a minimum of two vectors need to be detected in order to reconstruct the reference frame. Even if only two struts are visible, and they are partially obstructed, only the direction of each vector need be determined and the entire length of the strut need not be visible to the camera.

One or several struts can be omitted from the RM design to optimize visibility and reduce occlusions. For instance one or all RFs of a RM can be composed of only two struts.

Multiple RM's having different geometries and vector pair angles can be constructed and tracked simultaneously.

In another embodiment of the invention, the system features an algorithm running on the computer station 30 to help determine the position of the RM 100 and to increase the localization accuracy in the case of full or partial occlusion of individual markers (ie struts 120). The relationship between the position of the RM, or of a normal of a RF, and the line of sight of the camera can be used to calculate or predict the visibility of each face and of each strut. For example, when RF 130 is directly visible to one or both of the cameras, and the other RF 140 is occluded or beginning to become occluded, the algorithm can automatically ignore the vectors belonging to RF 140 and use primarily vectors belonging to RF 130 to determine the position. Weighted averaging can be used to reduce or eliminate the influence of partially visible RFs or individual struts.

In another embodiment, the bias errors (i.e., position and orientation error between the physical and measured strut centerlines) associated in determining the vectors of a RF when partial occlusion occurs are characterized according the relative position of the RM of RF and stored in the computer 30. The measured relationships are then used in a model to compensate for the biases during the actual measurements. The model can be an empirical one or an analytic one that takes as inputs the current measured position (with regards to orientation with respect to the camera and position of the RM in the measurement volume) and the correction factors required to compensate for the bias which depend on the position.

The RM 100 thus has 360° of visibility since at least one face is always in view as can be seen in FIG. 2. The computer can automatically detect the reference frames that belong to each RF 130. Multiple reference faces 130 can be used to optimize accuracy.

One of ordinary skill in the art will realize that solid polygonal shapes can also be used, which prevent the camera from seeing through each RF 130 so that the rear struts are not visible and do not confuse the system.

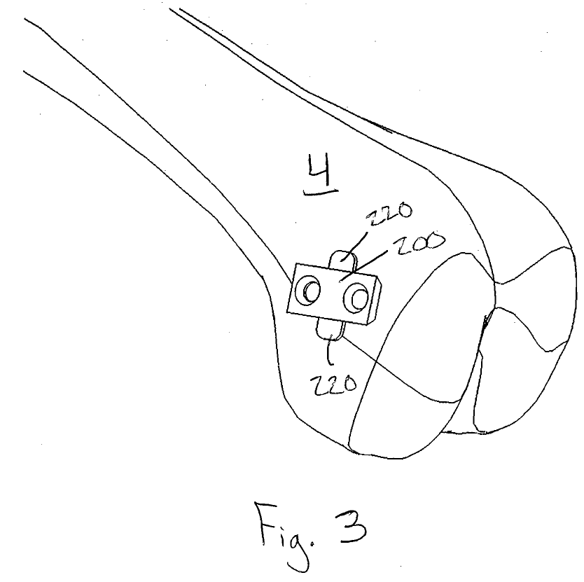

Referring now to FIG. 3, the RM 100 can be installed to the femur bone 4 using a low profile base 200, with a reproducible connection (RC) means 210 (FIG. 2), such as a dove tail joint with an axial stop. Tabs 220 on the sides of the base 200 help stabilize its position on the femur bone 4. The RC 210 can also be of a quick release type of connector, such as a snap connector, or can be another type of means that permits the RM 100 to be securely, yet releasably attached to the base 200. The low profile base 200 with reproducible fixation system 210 has advantages for minimally invasive surgery (MIS), where incisions are very small. The RM 100 can be removed at various stages during surgery so that the mobile incision window can be moved for example to the other side of the joint such as the lateral side. This can allow the surgeon to perform some actions on the other side, and during critical phases of the surgery when motion tracking is required, the RM 100 is put back on the base 200 via the RC 210. The RC 210 can also be made such that the RM 100 can be mounted in another predefined and known orientation with respect to the first orientation (for example, in the same plane but rotated at 90°). The system can automatically know which orientation the RM 100 is in, based on, for example, its orientation with respect to the camera reference frame. Alternatively, the protocol application could expect that the RM 100 is mounted in a certain orientation at a specific stage in the procedure. Alternatively, a specific point on the base 200 or on the bone 4 could be digitized with a pointer so that the system can compute if the orientation has changed from the initial one.

The support 230 of the RM 100 which serves to connect the reference marker 100 to the base 200 can be a curved shape so that it better exits outside the mobile incision window, which can be as small as a few centimeters or even smaller. The shape of the curve can also be optimized so that the RM 100 visible to the camera at all times, yet is not interfering with the surgeon or his tools while he operates on the patient as shown in FIGS. 1 and 2.

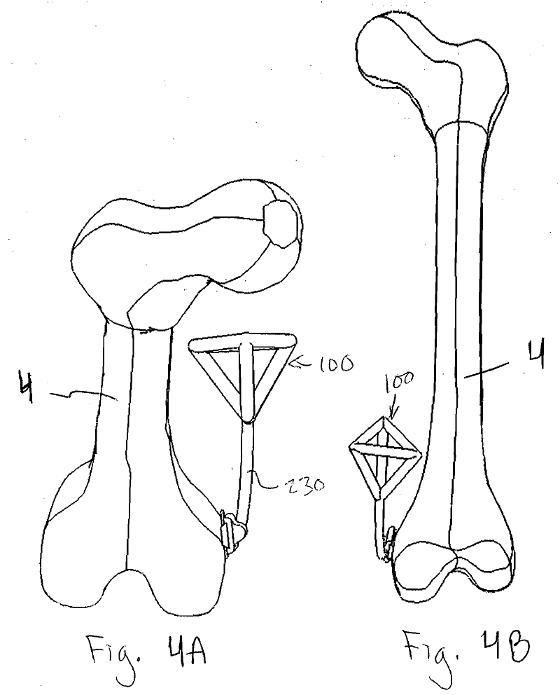

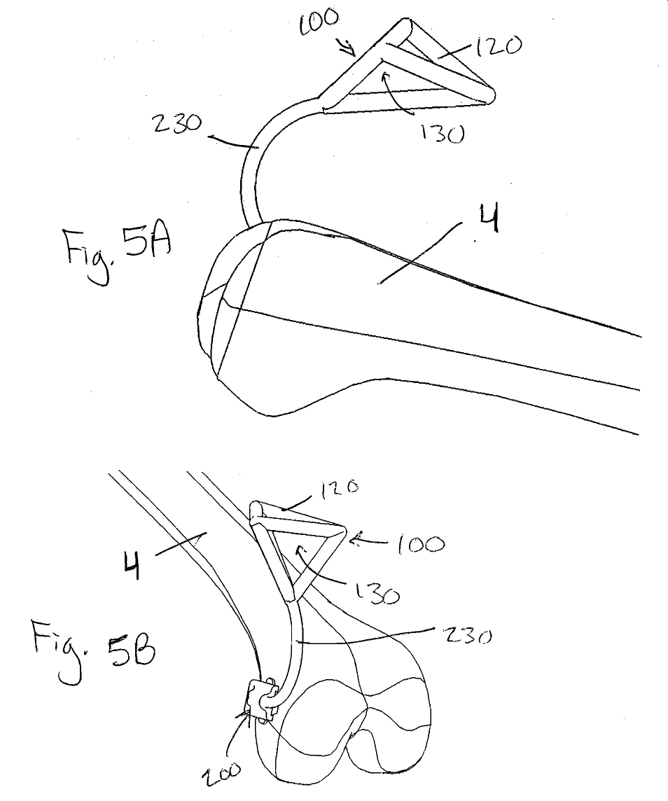

FIGS. 4A and 4B and FIGS. 5A and 5B show various other positions of the reference marker 100 attached to the bone 4 by means of the base 200. The curved nature of the support 230 is also shown and it will be appreciated that the structure of the support 230 and the reference marker 100 permits greater visibility thereof.

In sum, one exemplary reference marker 100 is a pyramid shaped rigid body with 360 degrees of visibility by means of using bars or struts 120, which are identified as lines in 2D camera images instead of using spheres, which are identified as circles in 2D camera images.

In addition, while the rigid body reference markers of the present invention can be attached to instruments to be navigated (e.g., digitization probes, drills, saws, drill-guides, planar probes, robots, robot arms, end effectors, etc.); the rigid bodies can also be directly integrated directly into the instrument. An example of a suitable instrument is set forth in commonly assigned U.S. patent application publication No. 2006/0149287 and an example of a robot is set forth in commonly assigned International Patent Application Publication No. WO 2006/106419, both of which are hereby incorporated by reference in their entireties.

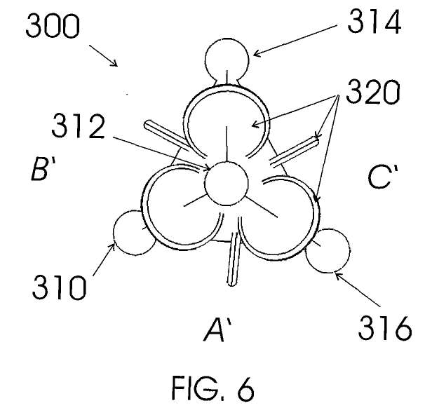

Now referring to FIGS. 6-8, a rigid body or reference marker (RM) 300 with 360 degrees of visibility is illustrated. The rigid body 300 includes four reflective spheres 310, 312, 314, 316 that are arranged in a pyramid formation such that the four spheres 310, 312, 314, 316 define four distinct planes or faces (References Faces, RF), namely, plane/face A′ which is defined by spheres 310, 312, 314; plane/face B′ which is defined by spheres 310, 312, 316; plane/face C′ which is defined by spheres 312, 314, 316 and plane/face D′ which is defined by spheres 310, 314, 316.

The spheres 310, 312, 314, 316 are arranged according to Polaris constraints so that they are compatible with the current stations of a system constructed by the present assignee. In one embodiment, the distances between the markers (sphere centers) is at least 50 mm with at least 5 mm of difference; however, other dimensions may be suitable depending upon the particular application or camera used. In particular, the inter-sphere distances can be much smaller if a higher resolution camera is used.

The rigid body 300 also includes disc-like dividers 320 that are provided to prevent the spheres 310, 312, 314, 316 from behind from overlapping the spheres 310, 312, 314, 316 in front when they are in the line with the line of sight of the camera or the like that is part of a system that detects an object to which the rigid body can be associated or attached.

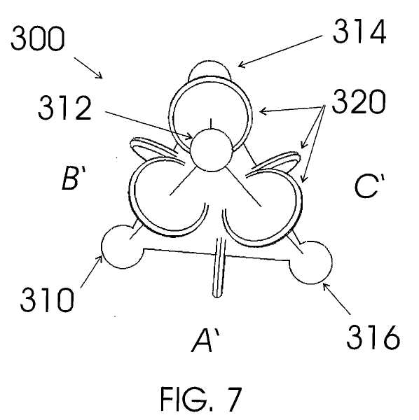

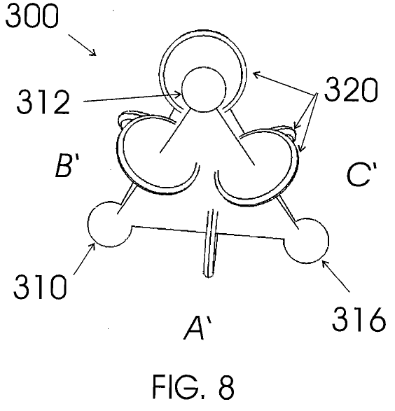

In the position of FIG. 6, spheres 310, 312, 314, 316 are clearly visible. The position of faces A, B, C can all be calculated. As seen in FIG. 7, as the rigid body 300 is rotated, the sphere 312 and the sphere 314 begin to fall in the same line of sight of the camera viewing plane. The occluding element (divider) 320 prevents the representation of the spheres from overlapping in the image acquired by the camera and keeps the image of the sphere 312 from becoming non-spherical. Thus, the accuracy of the calculation of the center point of the sphere is maintained. It will be appreciated that in FIG. 7, the sphere 314 is only partially visible. With additional rotation of the rigid body 300 to the position of FIG. 8, the occluding element 320 completely occludes the sphere 314 such that the sphere 314 is no longer visible.

The rigid body 300 is used with a computer based system (part of the detector system) that includes software that is designed for use with the rigid body 300. In particular, the software can include a switching algorithm such that when once face (A′, B′, C′, D′) of the rigid body 300 is visible with more than a certain degree from the line of sight, the camera ignores the other visible faces (A′, B′, C′, D′) which are near the limits of visibility or partially occluded and therefore, less accurate.

Alternatively, in certain positions, all four spheres 310, 312, 314, 316 can be used to provide more information when they are clearly visible, and the average of these four markers (spheres 310, 312, 314, 316) can be used to make a more stable reference frame.

To increase robustness, when one sphere 310, 312, 314, 316 is not visible (e.g., due to blood or water), the camera can detect the other three spheres 310, 312, 314, 316 even if they do not represent the plane that is most aligned with the camera line of sight. The rigid body 300 can also be used with a calibration system in that the relative 3D coordinates of the four spheres 310, 312, 314, 316 are stored in a file in the computer camera system. The accuracy of the position measurements can be determined as a function of the rotation angle in each plane with respect to the camera. Accuracy can decrease when spheres 310, 312, 314, 316 begin to become partially occluded. Based on this predetermined information, the camera can correct for any inaccuracies using the current measured position of the rigid body 300.

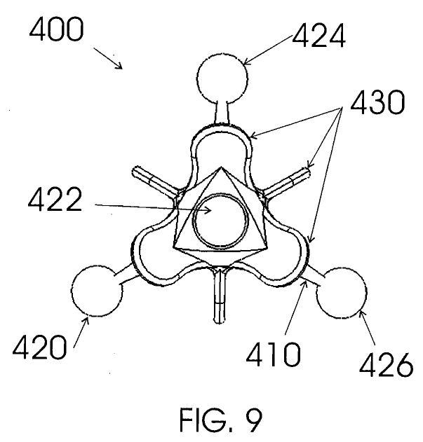

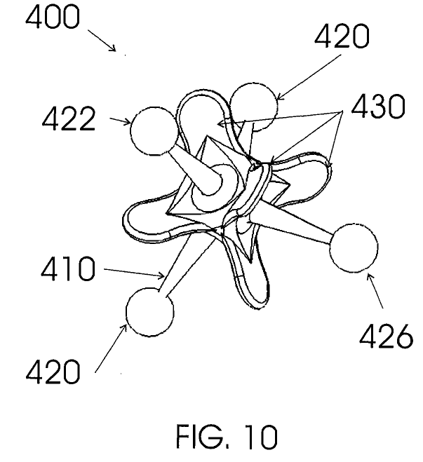

Referring now to FIGS. 9-10, a rigid body 400 according to another embodiment is illustrated. The rigid body 400 can be in the form of an organic tree-like structure with struts 410 extending out from the center area to each sphere 420, 422, 424, 426. The struts 410 can have a shape that is selected from the group consisting of cylindrical, conical, parabolic revolutions, etc. In addition, disk-like dividers 430 are provided to prevent the spheres 420, 422, 424, 426 from behind from overlapping the spheres 420, 422, 424, 426 in the front when they are in line with the line of sight of the camera or the like. The discs 430 can be joined as thin plates merging into the center stem area.

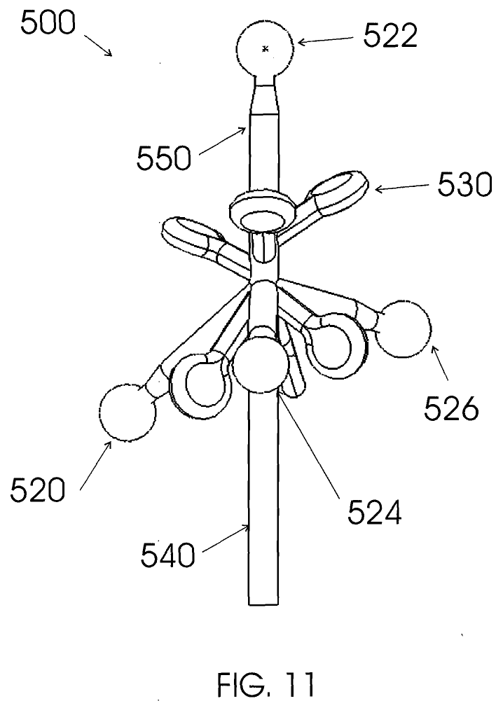

FIG. 11 shows a reference marker (RM) 500 with spheres 520, 524, 526, 522 having different distances from each other such that each reference face (RF) has a different geometry. Stem 540 provides a means for attachment to a bone, organ, or other object to be tracked. Branches or dividers 530 provide occlusions between all spheres. The divider size 530 can be optimized so as to block the sphere in the background just as it is beginning to overlap the sphere in the foreground. Dividers 530 and sphere support members 550 can be configured to radiate from the centroid area of the pyramid formed by the four spheres 520, 524, 526, 522.

As described previously, the system features an algorithm running on the computer station 30 to help determine the position of the RM 300 and to increase the localization accuracy in the case of full or partial occlusion of individual markers (i.e., spheres 310, 312, 314, 316, 420, . . . ) due to the dividers 430 or sphere supports 410 or fixation members 230. The relationship between the position of the RM, or of a normal of a RF, and the line of sight of the camera can be used to calculate or predict the visibility of each face and of each strut. For example, when RF A is directly visible to one or both of the cameras, and the other RF's B′ and C′ are occluded or beginning to become occluded, the algorithm can automatically ignore sphere 314 and use primarily spheres 310, 316, and 312 to determine the position of RM. Weighted averaging can be used to reduce or eliminate the influence of partially visible RFs or individual struts. In addition, the bias errors (ie position and orientation error between the physical and measured sphere centers) can be characterized according to the relative position of the RM or RF and stored in the computer 30 (i.e., as disc 320 begins the occlude sphere 314, the measured center of the sphere can shift upwards in the image away from the eclipsing edge and from the real physical sphere center). This measurement error can be measured and used in a model to compensate for the biases during the actual measurements. The model can be an empirical one or an analytic one that takes as inputs the current measured position (with regards to orientation with respect to the camera and the position in the measurement volume) and the correction factors required to compensate for the bias which depend on the position. This can be helpful for improving accuracy and visibility, particularly when another sphere is partially occluded, for example, due to blood.

As with the first embodiment, the rigid body 400 is of a pyramid design (so that it defines four distinct planes) and it can be made of plastic, light, injected modeled, disposable (no cleaning). Alternatively, a metallic (e.g., titanium) reusable design can also be used.

According to one aspect of the present invention, a passive marker is provided in which intermediate markers are used to define multiple faces, along with software algorithms for processing the data.

In view of the above, it will be seen that the several objects and advantages of the present invention have been achieved and other advantageous results have been obtained. It should be understood that any feature disclosed with respect to one arrangement of the invention can be equally applied to any other disclosed arrangement of the invention to yield additional benefits of the combined features.

Although this invention has been described with a certain degree of particularity, it is to be understood that the present disclosure has been made by way of example only and that numerous changes in the detailed construction and the combination and arrangement of parts may be resorted to without departing form the spirit and scope of the invention as hereinafter claimed.

What is claimed is:

1. A reference marker for use in a motion tracking system comprising:
a reference body being formed of a plurality of elongated bar members that are arranged according to a predetermined pattern, each bar being reflective to light, the plurality of bar members defining distinct reference faces that are arranged such that the reference marker has 360 degrees of visibility since at least one reference face is always in view and is visible to the motion tracking system.

2. The reference marker of claim 1, wherein the reference body has a pyramid shape that is defined by a triangular base and three triangular sides, each reference face being a triangular face that is defined by three bar members.

3. The reference marker of claim 1, wherein each bar member is a cylindrically shaped bar.

4. The reference marker of claim 1, further including:
a support coupled at one end to the reference body; and
a base that engages the support for attaching the reference body to a target object.

5. The reference marker of claim 4, wherein the support is an elongated bar member that has a curve formed along a length thereof.

6. The reference marker of claim 5, wherein the elongated bar member of the support has a cylindrical shape and is reflective to light.

7. The reference marker of claim 5, wherein the bar member of the support is curved between 45 degrees and 90 degrees.

8. The reference marker of claim 4, wherein the base is a low profile base that is attached to the target object that includes reproducible connection means that permits the reference body to be releasably, yet securely attached to the base.

9. The reference marker of claim 8, wherein the connection means comprises a dove tail joint with an axial stop.

10. The reference marker of claim 8, wherein the connection means comprises a quick release connector in the form of a snap-fit type connector that permits the reference body to be snap fittingly attached to the base.

11. A rigid body with 360 degrees of visibility comprising:
four reflective spheres arranged in a pyramid formation such that four distinct reference planes are defined, each reference plane being defined by three spheres; and
disc shaped dividers arranged to prevent spheres from behind from overlapping the spheres in the front when the spheres from behind are in line with the line of sight of a detector.

12. The rigid body of claim 11, wherein the detector comprises a camera.

13. The rigid body of claim 11, wherein the reference plane has a triangular shape.

14. The rigid body of claim 11, wherein the four spheres are attached to a pyramid shaped reference body and the disc shaped dividers are attached to the pyramid shaped reference body at locations between two spheres.

15. The rigid body of claim 11, wherein each of the spheres is positioned at a distal end of an elongated strut that extends from a reference body that has an amorphous shape, the dividers being integrally formed with the reference body as a single structure.

16. The rigid body of claim 15, wherein the strut comprises a post.

17. A motion tracking system comprising:
a position measuring device for detecting the position and orientation of an object by tracking the relative movement of a reference marker attached to the object;
a computer that is configured to determine and track positions of the reference marker;
wherein the reference marker is constructed so that it includes four distinct reference faces that are arranged such that the reference marker has 360 degrees of visibility since at least one reference face is always in view and is visible to the position measuring device.

18. The system of claim 17, wherein the object is selected from the group consisting of a bone, a tool and a pointer.

19. The system of claim 17, wherein the position measuring device comprises an optical tracking system and the reference marker is reflective to light.

20. The system of claim 17, wherein the reference marker includes a reference body formed of a plurality of elongated bar members that are arranged according to a predetermined pattern, each bar being reflective to light, the plurality of bar members defining the four distinct reference faces that are arranged such that the reference markers has 360 degrees of visibility.

21. The system of claim 20, wherein the reference body has a pyramid shape that is defined by a triangular base and three triangular sides, each reference face being a triangular face that is defined by three bar members.

22. The system of claim 20, wherein the position measuring device comprises an optical tracking system including at least one camera and the bars are identified as lines in 2D camera images.

23. The system of claim 17, wherein the computer is configured such that once one of the reference faces is visible more than a predefined number of degrees from a line of sight of the position measuring device, the device ignores any other visible reference faces which are near the limits of visibility or are partially occluded and less accurate.

24. The system of claim 17, wherein the reference marker comprises a rigid body having a pyramid shape with four reflective spheres arranged in pyramid formation such that the four spheres defines four distinct faces, each face being defined by three spheres.

25. The system of claim 24, wherein the reference marker includes disc-shaped dividers arranged to prevent spheres from behind from overlapping the spheres in the front when the spheres from behind are in line with the line of sight of the position measuring device.

26. The system of claim 24, wherein the computer is configured such that when one reflective sphere is not visible to the position measuring device, the measuring device is capable of detecting the other three reflective spheres even if the three other reflective spheres do not represent the reference face that is most aligned with the line of sight of the position measuring device.

27. The system of claim 25, wherein the dividers are constructed and arranged so that when the rigid body is rotated, one divider prevents the representation of two of the spheres from overlapping in an image acquired by a camera of the position measuring device, thereby keeping the image of one sphere from becoming non-spherical.

28. The system of claim 17, wherein the reference marker includes a main support coupled at one end to the reference marker and a base that engages the support for attaching the reference marker to the object.

29. The system of claim 28, wherein the main support is an elongated bar member that has a curve formed along a length thereof.

30. The system of claim 29, wherein the elongated bar member of the main support has a cylindrical shape and is reflective to light.

31. The system of claim 30, wherein the bar member of the main support is curved between 45 degrees and 90 degrees.

32. The system of claim 28, wherein the base is a low profile base that is attached to the target object that includes reproducible connection means that permits the reference marker to be releasably, yet securely attached to the base.

33. The system of claim 24, wherein when the reference marker is in a position where all four spheres are visible to the position measuring device, the computer is configured to average information obtained from the positions of the four spheres to produce an optimal reference frame, each reference face being seen as a reference frame detected by the position measuring device.

34. The system of claim 20, wherein if one bar member is occluded due to the orientation of the reference marker with respect to the position measuring device, the reference face defined by the one bar member has a lower weighting than one or more reference faces that are more visible to the position measuring device.
